(19) United States
(12) Patent Application Publication
(10) Pub. No.: US 2025/0221346 A1
(43) Pub. Date: Jul. 10, 2025

(54) METHOD FOR ACCELERATING PLANT CROP CYCLE

(71) Applicant: Monsanto Technology LLC, St. Louis, MO (US)

(72) Inventors: Addi Akaddar, Gien (FR); Ying Guo, Ballwin, MO (US); Huachun Larue, Chesterfield, MO (US); Huai Wang, Chesterfield, MO (US); Miin-Feng Wu, Ballwin, MO (US)

(21) Appl. No.: 18/984,004

(22) Filed: Dec. 17, 2024

Related U.S. Application Data
(60) Provisional application No. 63/617,910, filed on Jan. 5, 2024.

(51) Int. Cl. A01G 7/04; A01H 3/02

(52) U.S. Cl. CPC A01G 7/045; A01H 3/02

(57) ABSTRACT

Methods for decreasing flowering time and accelerating crop cycle in plants and increasing the number of breeding cycles per year in such plants are provided. The methods comprise using far-red light alone or in combination with additional external signals such as photoperiod in order to decrease flowering time and accelerate seed to seed crop cycle. Also provided are methods for bypassing vernalization requirements in winter annuals.

### CROSS-REFERENCE TO RELATED APPLICATIONS

This application claims priority to U.S. Provisional Patent Application No. 63/617,910, filed Jan. 5, 2024, the entire disclosure of which is incorporated herein by reference

### FIELD OF THE INVENTION

The present invention relates to the fields of plant breeding and crop science. More specifically, the present invention relates to methods for decreasing flowering time using far-red light and increasing the number of breeding cycles per year in such plants.

### BACKGROUND OF THE INVENTION

Vernalization describes an over-winter response in which a prolonged exposure to cold temperature is required for flowering to occur in spring. The requirement of vernalization is a critical survival mechanism that protects the reproductive organs of certain plants by allowing them to form only when temperature is optimal. Research in this area has demonstrated that artificial, winter-like conditions can trigger winter annual crops to flower in a controlled environment (CE). Furthermore, previous studies in the model plant Arabidopsis have demonstrated that the vernalization-mediated flowering response is controlled by a MADS-box transcription factor FLOWERING LOCUS C (FLC). Before vernalization, FLC binds directly to the first intron of a flowering activator FLOWERING LOCUS T (FT) to repress FT expression. Upon exposure to low temperature, transcription of FLC is first turned off by anti-sense RNA COOLAIR. Prolonged cold exposure subsequently activates epigenetic regulators to deposit silencing histone modifications first around the transcription start site then eventually to the whole gene body of FLC throughout winter. This requirement of long cold exposure prevents plants from flowering during sudden temperature spikes in winter, and the eventual stable silencing of FLC after winter safeguards successful flower formation when spring arrives. To date, all published literature attempting to shorten winter annual crop cycle time has been based on the assumption that vernalization is a requirement for flowering, even under CE.

It has therefore been standard practice for several decades to incubate plants for multiple months at a temperature of around 4° C. under a short-day photoperiod, to mimic the necessary vernalization conditions and thus induce flowering of winter annuals. The requirement for an extensive artificial vernalization treatment has become the biggest hurdle in accelerating crop cycle time for winter annual crops such as winter oilseed rape (WOSR) in CE-based commercial breeding pipelines. Although researchers have attempted to shorten the winter crop cycle time by optimizing vernalization conditions, so far step-change progress has not been achieved in winter annual crops. Moreover, the principle that vernalization is required to trigger winter annuals to flower has never been directly challenged. Increasing breeding cycles per year, especially for winter annuals, is essential towards rapidly accelerating genetic gains for the development of improved, desirable germplasm. Therefore, a continuing need exists in the art to develop novel methods to decrease flowering time, accelerate seed to seed crop cycle, and thus accelerate plant breeding cycles effectively and efficiently.

### SUMMARY OF THE INVENTION

In one aspect, the invention provides a method for decreasing flowering time in a plant. In one embodiment, the method comprises the steps of: (a) obtaining a juvenile plant; and (b) treating the juvenile plant with far-red light to induce a flowering response in said plant. In certain embodiments, said treating is carried out when the plant is from about 1 day to about 7 weeks from germination; or is carried out at a temperature of from about 4° C. to about 30° C. In specific embodiments, said treating is carried out at a temperature from about 6° C. to about 15° C.; or is carried out at a temperature from about 15° C. to about 30° C. In some embodiments, said treating is carried out for about 1 to about 7 weeks; or is carried out at a temperature of about 10° C. for about 1 to about 5 weeks. In further embodiments, said treating is carried out in the presence of visible light or photosynthetic active radiation (PAR). In some embodiments, growing the plant under far-red light accelerates plant development and/or seed maturation as compared to a control plant lacking said treatment. In other embodiments, the method further comprises growing the plant under far-red light at a temperature of from about 10° C. to about 34° C.; or growing the plant under far-red light is carried out at a temperature of about 10° C. to about 25° C. In yet other embodiments, said treating comprises growing the plant under a photoperiod from 16 hr to 24 hr. In one embodiment, the photoperiod is at least 20 hr.

In other embodiments, the methods provided herein comprise treating the plant with far-red light at a red light to far-red light ratio between 0.01 to 0.8. In further embodiments, the far-red light comprises an intensity of about 50 μmoles/m·s to about 800 μmoles/m·s. In certain embodiments, the far-red light is applied continuously; non-continuously; or is applied from about 1 hr to about 24 hr per day prior to flowering. In a further embodiment, the plant is treated with far-red light for about 7 to about 60 days prior to flowering. In some embodiments, the flowering response is induced without vernalization. In other embodiments, the flowering response is initiated in less than about 45 days from germination; or in less than about 7 days from germination. In some embodiments, the method further comprises allowing the plant to develop at least a first flower. In other embodiments, far-red light is applied to the plant during seed maturation. In still further embodiments, the method further comprises crossing said plant with itself or a second non-isogenic plant. In specific embodiments, the method further comprising harvesting a seed from the plant. Plants that may be used in the methods described herein include vernalization dependent and vernalization independent plants. Non-limiting examples of types of plants that may be used in the methods disclosed herein include wheat, barley, rye, oat, oilseed rape, bok choy, cabbage, cauliflower, collards, broccoli, brussels sprouts, kale, kohlrabi, rutabagas, turnips, sugar beet, corn, soybean, and cotton. In specific embodiments, the plant is a winter oilseed rape plant or a winter wheat plant.

In another aspect, a method of bypassing vernalization dependent flowering in a plant is provided. In one embodiment, the method comprises the steps of (a) obtaining a juvenile plant; and (b) treating the juvenile plant with far-red light to induce a flowering response in said plant; wherein said plant is a vernalization dependent plant and wherein said treating is carried out without vernalization prior to said flowering response. In some embodiments, the method further comprises vegetatively propagating the plant. In other embodiments, the flowering response results in at least a first floral bud forming less than about 60 days after germination of the plant. In a particular embodiment, the method further comprises harvesting a seed resulting from the flowering response. In certain embodiments, the method further comprises crossing the plant with itself or a second plant to produce a progeny plant. In further embodiments, the method comprises harvesting a seed resulting from said crossing.

In still yet another aspect, a method of accelerating crop cycle time in a plant is provided, the method comprising: (a) obtaining a juvenile plant; and (b) treating the juvenile plant with far-red light to induce a flowering response in said plant; (c) allowing the plant to develop at least a first floral bud; wherein the first floral bud is visible in less than about 60 days from germination. In some embodiments, the method further comprises growing the plant under far-red light, wherein growing the plant under far-red light accelerates plant development. In other embodiments, the method further comprises growing the plant under far-red light following development of at least the first floral bud, wherein growing the plant under far-red light accelerates seed maturation. In one embodiment, the method further comprises harvesting a seed from the plant. In other embodiments, said plant is a vernalization dependent plant.

In yet another aspect, a method for decreasing seed to seed cycle time in a plant, the method comprising: (a) obtaining a juvenile plant; (b) exposing the juvenile plant to a temperature from about 4° C. to about 15° C. to induce a flowering response in said plant; and (c) growing the plant at a temperature from about 16° C. to about 34° . In some embodiments, the step (b) further comprises treating the plant with far-red light; step (c) further comprises treating the plant with far-red light; or steps (b) and (c) further comprise treating the plant with far-red light. In other embodiments, treating the plant with far-red light accelerates plant development or seed maturation as compared to a control plant lacking said treatment. In certain embodiments, exposing the plant to a temperature from about 4° C. to about 15° C. is carried out for about 1 to about 7 weeks. In further embodiments, the plant is exposed to a temperature from about 10° C. to about 15° C. for about 4 weeks. In still further embodiments, growing the plant is carried out at a temperature of about 22° C.

### BRIEF DESCRIPTION OF THE DRAWINGS

FIG. 1: Panel A shows a close-up view of a representative macrografting junction formed between a WOSR scion and a Canola root stock. Unvernalized WOSR shoot (scion) are grafted onto a decapitated Canola plant (rootstock). The junction is circled. Panels B and C show representative time series images from macrografted plants. Numbers underneath each plant indicate days after planting of the unvernalized WOSR scion. Pedigree names in the upper right corners are indicated as scion/rootstock, e.g. CR72/CK6671 (Panel B) and CB2036/CK6671 (Panel C). Unvernalized WOSR plants of the same scion pedigree are shown as the no treatment (No Trt) control.

FIG. 2: Panel A shows a close-up view of representative micrografting and micrografting junctions. Canola shoot is first grafted onto unvernalized WOSR hypocotyl by micrografting. After the grafted plant matures, an unvernalized WOSR scion is grafted onto the decapitated shoot of the micrografted plant. Graft junctions are circled. Panel B shows representative images of flowering plants from the grafting procedures shown in Panel A. Pedigrees are indicated as their relative section in the grafted plant. No treatment (No Trt) unvernalized WOSR plants is shown as a control. Days after planting of the last grafted unvernalized scion at the time of photographing is indicated in the lower right corner.

FIG. 3: Shows representative images of winter and spring oilseed rape treated with far-red light on day 3 after planting. Panel A shows winter OSR—enlarged meristem indicative of reproductive transition after 3 days under FR (+FR d3, asterisk). Floral primordia are initiated after 12 days under FR (+FR d12, arrowheads). 15-day-old no treatment (No Trt) plant of the same age as +FR d12 is shown on the left as a comparison. Panel B shows spring OSR (Canola)—flowers are fully differentiated after 8 days under FR (+FR d8). 11-day-old no treatment (No Trt) plant of the same age is shown as a comparison (FIG. 1B). Scale bars for plants: 5 cm. Scale bars for meristem: 0.2 mm.

FIG. 4: Shows diverse germplasm pools can flower under a representative FR treatment protocol. Panel A shows a bar graph representing average bud visible days after planting; each dot represents one plant. Panel B shows representative images of flowering plants treated with FR (right plant); and untreated plants (left plant). Pedigree names are indicated in the upper left corner. Scale bars: 15 cm.

FIG. 5: Shows bar graphs representing expression levels of two FT or FLC paralogs obtained by quantitative real-time PCR. Cotyledons from 4 representative WOSR pedigrees were harvested after 1 week with or without FR treatment. Panel A shows relative expression of FT paralog and panel B shows relative expression of FLC paralog. Expression levels from 3 replicates ±SEM relative to no FR treatment control (No Trt) are shown.

FIG. 6: Shows histone modification H3K27me3 at the FLC loci is not required for FR-induced flowering. Panel A shows H3K27me3 fold enrichment levels at two representative WOSR FLC paralogs from CR134 plants treated with 10° C. for 4 weeks (10° C.), no treatment (No Trt) and FR-treated (FR) for 14 days starting on day 3 after planting as measured by chromatin immunoprecipitation (ChIP) followed by quantitative PCR. Schematic relative locations for genomic fragments amplified by qPCR are indicated. Panel B shows representative FT and FLC paralog expression levels in plants from no treatment (No Trt) and FR-treated (FR) at the time of harvesting for ChIP. Expression levels from 3 replicates ±SEM relative to plants treated at 10° C. for 4 weeks are shown.

FIG. 7: Shows low temperature incubation and FR treatment create synergistic effects in accelerating flowering. Panel A shows low temperature treatment followed by FR (10° C.→22° C.+FR) reduces average bud visible time of 4 representative pedigrees to 7-9 weeks compared to 11 weeks using a conventional vernalization protocol (4° C.); each dot represents one plant. Panel B shows plants incubated at low temperature then moved under FR at standard growth temperature. Enlarged meristem indicative of reproductive transition is visible after 1 day under FR (+FR d1, asterisk). Floral primordia are initiated after 4 days under FR (+FR d4, arrowhead). Flowers are fully differentiated after 7 days under FR (+FR d7). 33-day-old no treatment plants (No Trt) of the same age as +FR d7 plant is shown as a comparison. Panel C shows adding FR (+FR) after low temperature treatment shows synergistic effects in flowering acceleration. Two representative pedigrees are shown. Pedigree names are indicated in the upper left corner. Images are taken 3 weeks after FR treatments. Meristems from no treatment (No Trt) plant of the same age is shown on the left. Scale bars for plants: 5 cm. Scale bars for meristem: 0.2 mm.

FIG. 8: Shows FR treatment accelerates flower development across different temperatures. Panel A shows representative images of the reproductive transition or flower formation of three WOSR pedigrees (CR134, CR598 and CS844) is visible after 4 weeks under 10° C.+FR (asterisk and arrows). 4 days after returning to 22° C. plus FR, reproductive transition has occurred in plants treated with 10° C. without FR (asterisks). On the same day, more advanced flower development is seen in plants treated with 10° C.+FR (arrows). Scale bars: 0.2 mm. Panel B shows a comparison of average bud visible time of plants shown in panel A to plants without low temperature treatments. Adding FR during 10° C. treatment further accelerates bud visible time. Panel C shows average bud visible time from pedigree CR134 under different temperatures with or without FR treatments. Supplementing FR during low temperature treatment further reduces bud visible time. Continuous FR treatment with background lighting for 24 hr daily shows the most significant reduction in bud visible time. Numbers above bars in panels B and C indicate average bud visible days after planting. Panel D shows expression levels of FT paralog FT.C6 and FLC paralog FLC.A10 with or without FR treatment. Under low temperature FLC is reduced by half compared to the treatment starting timepoint (d3, 3 days after planting). Adding FR under low temperature significantly upregulates FT expression. 1 w-3 w: treatment duration 1 week to 3 weeks. Expression levels from 3 replicates ±SEM relative to DNA fragment of known concentration are shown.

FIG. 9: Shows FR treatment coupled with >16 hr photoperiod can further accelerate flowering. Images of WOSR pedigree CS844 are shown as a representative example. Under 16 hr photoperiod CS844 produces floral buds on 55 or 60 days after planting (dap), compared to 22-21 dap under 22 hr photoperiod (see also FIG. 4; Panel A). Panels A and B show shoot apical meristems on 15 days after planting (dap) and 22 dap under 16 hr photoperiod, respectively. Panels C and D shows floral buds on 15 dap and 22 dap under 22 hr photoperiod, respectively. Panel E shows CS844 plants under 16 hr or 22 hr photoperiod on 47 dap. Scale bars: 0.2 mm for Panels A-C; and 5 cm for Panel E.

FIG. 10: Shows FR treatment with >16 hr photoperiod promotes stronger FT induction. Panels A and B show expression change of FT paralogs A2 and C6 from WOSR pedigrees CR134 (Panel A) and CS844 (Panel B) 19 days after FR treatment as measured by quantitative real-time PCR. Shown are mean of 3 replicates ±SEM. Expression levels are normalized to no FR treatment control of the same leaf number. Tissues were harvested at ZT21 or ZT15 for 22 hr, 16 hr photoperiods, respectively.

FIG. 11: Shows FR supplementation in winter wheat accelerates winter wheat crop cycle time. % lines responded is calculated by number of lines flowered/number of lines tested. Cycle time folds acceleration is calculated based on seed-to-seed time compared to conventional method. In total, 32 diverse elite winter wheat lines were represented in these experiments.

FIG. 12: Shows vegetative cuttings from FR-treated WOSR continue to flower. Cuttings from FR-treated WOSR inflorescences are treated with rooting hormones at the cut site then inserted into peat moss-based plugs until roots are initiated. The resulting plantlets are transplanted into nursery pots with peat moss-based soilless medium until flowering. Flowering vegetatively propagated plants from four representative pedigrees are shown. Pictures are taken 3 to 4 weeks after newly formed floral buds are visible from the top.

FIG. 13: shows the effect of FR treatment at varying plant ages. Panel A shows average floral bud visible time achieved across different plant age when FR treatment was first initiated, as shown as days after planting (dap), data from five representative pedigrees were shown. Seedlings were germinated at constant 22° C., 22 hr photoperiod under broad spectrum LED set to 300 μmol m^{31 2} s⁻¹. Seedlings were moved under FR set to 22 hr photoperiod at different dap until floral buds were visible from the top of canopy. All WOSR plants produced visible buds and flowers without vernalization. Panel B shows FR treatment initiated on plants that were up to 28 days after planting (dap) can promote flowering without vernalization (pedigree CR134). Seedlings were germinated at constant 24° C. with constant light (24 hr) set to same intensity as in Panel A. Seedlings were moved under FR set to identical photoperiod (24 hr) at different age as indicated with dap.

FIG. 14: Shows R/FR ratio effect on flowering acceleration of FR. Pedigree CR134 seedlings were germinated for 3 days at 24° C., under constant light (24 hr) with broad spectrum LED set to 300 μmol m⁻² s⁻¹ then moved under FR under identical temperature (24° C.) and photoperiod (24 hr). FR intensity was adjusted to 17% of standard treatment condition while red light intensity remained constant to achieve different R/FR ratio. Increasing R/FR ratio from 0.3 to 0.6 significantly delayed floral bud visible time by 17 days in the absence of vernalization. Numbers above bars indicated mean of 4 plants.

FIG. 15: Shows the photoperiod effect of FR treatment. Panel A shows seedlings of 4 representative pedigrees germinated for 3 days at 24° C., under constant light (24 hr) with broad spectrum LED set to 300 μmol m⁻² s⁻¹. 3-day-old seedlings were moved under FR at 24° C. with different photoperiod settings. Results showed that plants overall flower faster under longer photoperiods with the fastest bud visible time occurring under 24 hr constant light. N.F.: No Flower at 70 dap. Panel B shows a diagram illustrating the 10+10 hr settings with FR turned off for 2 hr in the middle of the day. With all other photoperiod setting, WL and FR are synchronized with same duration of illumination.

FIG. 16: Shows FR effect on accelerating wheat flowering. Panel A shows the phenotypic response of winter wheat cultivars to different growth conditions without going through cold temperature induced vernalization. Line1: B-PW48-MS390; line2: B-CW47-AM208; Line3: B-PW17-DM396; and Line4: B-NE48-XH318. Germinated seedlings were grown in 3 different conditions: Control, Speed flowering, and Ultra Speed flowering. Photos were taken when plants were 116 days from sowing. All three growth conditions were set as 23/18° C. (D/N), broad spectrum LED with light intensity of 600 μmol/m2/sec. 1) Control flowering (CF) is set to 16/8 hr light/dark cycle; 2) Speed flowering (SF) is set as 22/2 hr light/dark cycle; and 3) Ultra speed flowering (USF) set as 22/2 hr light/dark cycle plus supplemental far-red light, R/FR ratio of about 0.3 in this experiment. Panel B shows the phenotypic response of spring wheat cultivars to CF, SF, and USF growth conditions. DAMI9-7022 represent typical spring lines, flowers early. PAMI9-4018 represent late flowering spring lines. Photos were taken when plants were 40 days from sowing.

### DETAILED DESCRIPTION

“Genetic gain” is defined as the improvement in average genetic value in a population or the improvement in average phenotypic value due to selection within a population over cycles of breeding. Genetic gain can only be realized from executing at least one cycle of breeding; and, in practice, multiple breeding cycles are necessary to realize genetic gains with respect to one or more heritable traits. Although many factors can affect genetic gain (e.g. heritability, phenotype variation, and selection intensity) crop cycle time is the single most important factor in accelerating genetic gain within a population.

Reducing crop cycle time can therefore significantly accelerate genetic gain. However, current protocols for reducing crop cycle time are limited, especially for winter annual crops such as winter oilseed rape. In particular, the bottleneck in accelerating winter annual crop breeding cycles lies in the natural requirement to induce a plant's flowering process via exposure to the prolonged cold of winter, or by exposure to an artificial equivalent using a controlled environment. This period of long cold exposure, generally referred to as vernalization, is believed to be required for flowering in winter annuals and other vernalization-dependent plants. Previous studies have attempted to shorten the winter crop cycle time by optimizing vernalization conditions; however, step-change progress has not been achieved in winter annual crops. See, e.g. Cha, J. K. et al. Mol Plant 15, 1300-1309 (2022) and Song, Y. et al. Plant Biotechnol J 20, 13-15 (2022). Thus, there is a continuing need for discovery and development of new strategies for reducing crop cycle time in winter annual crops and other vernalization-dependent crop species.

The present disclosure overcomes the limitations of the prior art by providing methods for decreasing flowering time in a plant comprising treating a juvenile plant with far-red light to induce a flowering response. In particular, the present disclosure demonstrates that when far-red light (FR) treatment is applied to young plants, flowering can occur in a temperature agnostic manner, bypassing the requirement of prolonged cold temperature treatment (4-6° C., for 8 weeks) that has been the standard practice for winter annual crops (e.g. winter oilseed rape) for decades. The methods described herein can lead to a 2-3 fold reduction in flowering time across diverse germplasms yielding significant improvements toward accelerating winter annual breeding cycle time and genetic gain. The present disclosure further demonstrates that FR treatment across different growth temperatures can activate the expression of flowering promoter FT without transcription or epigenetic silencing of the flowering inhibitor FLC, breaking the current paradigm that FLC silencing is required for subsequent FT activation. Moreover, when FR is applied with low temperature treatment, a simultaneous reduction of FLC and upregulation of FT creates a synergistic effect in flowering acceleration. The ability to produce these desirable effects using the methods described herein offers unique benefits not otherwise available in the art; and enables novel breeding workflows that significantly accelerate genetic gain.

The embodiments described herein therefore relate to methods for decreasing flowering time in a plant by treating the plant with far-red light to induce a flowering response. Also described are methods for bypassing vernalization dependent flowering in a plant, including vernalization-dependent plants. In certain applications, a far-red light (FR) treatment is provided to a plant at the juvenile phase to induce flowering. In certain embodiments, such FR treatment is carried out when the plant is from about 1, 2, 3, 4, 5, 6, 7, 8, 9, 10, 11, 12, 13, 14, 15, 16, 17, 18, 19, 20, or 21 days from germination. In other embodiments, the FR treatment is carried out when the plant is from about 1, 2, 3, 4, 5, 6, 7, 8, 9, or 10 weeks from germination. In still other embodiments, FR treatment can be carried out for about 5, 6, 7, 8, 9, 10, 11, 12, 13, 14, 15, 16, 17, 18, 19, 20, 25, 30, 35, 40, 45, 50, 55, or about 60 days prior to flowering. In still further embodiments, FR treatment can be carried out when the plant is from about 1, 2, 3, 4, 5, 6, 7, 8, 9, 10, 11, 12, 13, 14, 15, 16, 17, 18, 19, 20, 21, 22, 23, 24, 25, 26, 27, or 28 days from germination and continue until seed maturation. In specific embodiments, FR treatment can be carried out during or following development of at least a first floral bud to accelerate flower development and seed maturation, as compared to an appropriate control plant lacking said treatment. Such FR treatment can be continuous or non-continuous.

Several embodiments of the invention relate to a method comprising: (a) obtaining a juvenile plant; and (b) treating the juvenile plant with far-red light to induce a flowering response in said plant. Treating the juvenile plant with far-red light can by carried out at a range of temperatures. For example, treating can carried out at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22°° C., 23° C., 24° C., 25° C., 26° C., 27° C., 28° C., 29° C., or 30° C. As described herein, such FR treatment can be carried out at one more temperatures for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 days. In specific embodiments, the FR treatment is carried out for about 1, 2, 3, 4, 5, 6, or 7 weeks or until seeds of the plant mature.

Several embodiments of the invention further relate to growing the plant under far-red light. For example, the methods provided herein can include growing the plant under far-red light at a temperature of about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., or 34° C. In specific embodiments, the plant is grown under far-red light at a temperature of about 20° C., 21° C., 22° C., 23° C., 24° C., or 25° C. Growing the plant under far-red light as described herein can accelerate plant development, including but not limited to, accelerating the development of floral buds, and accelerating the development and maturation of seeds. Growing the plants according to the methods described herein may also be carried under a photoperiod of about 16 hr, 17 hr, 18 hr, 19 hr, 20 hr, 21 hr, 22 hr, 23 hr, or 24 hr. As described herein, growing the plants under a photoperiod greater than 16 hr, e.g. at least about 20 hr, can further decreasing flowering time as compared to photoperiods less than or equal to 16 hr.

Far-red light has a wavelength of approximately 700 nm to 750 nm with a peak wavelength at 730 nm, whereas red light has a wavelength of approximately 625 nm to 740 nm with a peak wavelength at 660 nm. In some embodiments the methods provided herein comprise treating a plant with far-red light, wherein the red light to far-red light ratio is about 0.01, 0.02, 0.03, 0.04, 0.05, 0.06, 0.07, 0.08, 0.09, 0.1, 0.2, 0.3, 0.4, 0.5, 0.6, 0.7, 0.8, or more.

The methods for decreasing flowering time provided herein include treating plants continuously with far-red light or non-continuously with far-red light. For example, in some embodiments far-red light is applied to the plant for about 1, 2, 3, 4, 5, 6, 7, 8, 9, 10, 11, 12, 13, 14, 15, 16, 17, 18, 19, 20, 21, 22, 23, or 24 hr per day. In addition, FR treatment can be carried out in the presence of supplementary light. For example, in the presence of visible light or in the presence of a range of wavelengths within the visible spectrum or in the presence of sources of photosynthetic active radiation. In certain embodiments, the far-red light intensity may range from about 50 μmoles/m·s to about 800 μmoles/m·s, including about 55, about 60, about 65, about 70, about 75, about 80, about 85, about 90, about 100, about 150, about 200, about 250, about 300, about 350, about 400, about 450, about 500, about 550, about 600, about 650, about 700, about 750, or about 800 μmoles/m·s.

Sources for applying FR are known in the art. See, e.g. Filippos Bantis, et al. Current status and recent achievements in the field of horticulture with the use of light-emitting diodes (LEDs). Scientia Horticulturae, Volume 235; 437-451 (2018). For example, far-red light may be provided to the plants with the use of artificial lights. Non-limiting examples of artificial lighting include, the use of a 1000-watt metal halide portable light tower placed over the plants. Other types of light such as high-pressure sodium or LED may be provided, as well. In some embodiments, far-red light treatment conditions may be provided to a field-grown plant by exposing the field-grown plant to artificial light.

Some embodiments described herein relate to decreasing seed to seed cycle time in a plant by exposing the plant to a temperature from about 4° C. to about 15° C. to induce a flowering response in said plant; and growing the plant at a temperature from about 16° C. to about 34° C. Such methods may be carried out with or without treating the plant with far-red light. In certain embodiments, the plant may be treated with far-red light during exposure to a temperature from about 4° C. to about 15° C.; the plant may be treated with far-red light during growth at a temperature from about 16° C. to about 34° C.; or during both steps. In specific embodiments, treating the plant with far-red light at a temperature from about 4° C. to about 15° C. synergistically accelerates a flowering response in said plant.

The methods described herein solve a variety of crop cycle time concerns and can be utilized in the research, regulatory, breeding, and commercial phases of product development. For example, the amount of time required to produce flowering can be decreased and the number of crop cycles completed can be increased depending on the need in a particular product development phase. Utilizing the methods described herein, it is possible to accelerate genetic improvement especially in vernalization dependent crop species. It is also possible to apply the methods described herein to a wide range of breeding paradigms and to improve operational efficiency and flexibility. This provides case in plant breeding by enabling breeders to produce crosses of plants on significantly faster timelines, that would normally not be possible due to the requirements for vernalization. These advantages to the breeding process can ultimately lead to faster genetic gains and more rapid launch of new commercial germplasms compared to the historical methods, especially in winter annual crop species. For example, in certain applications of the methods described herein, the flowering response is initiated in less than about 40, 41, 42, 43, 44, or about 45 days from germination. In specific embodiments, the flowering response is initiated in less than about 4, 5, 6, 7, 8, 9, 10, 11, 12, 13, 14, or 15 days from germination.

Several embodiments described herein relate to a method of bypassing vernalization dependent flowering in a plant, the method comprising: (a) obtaining a juvenile plant; and (b) treating the juvenile plant with far-red light to induce a flowering response in said plant; wherein said plant is a vernalization dependent plant and wherein said treating is carried out without vernalization prior to said flowering response. The ability to bypass the requirement for vernalization in a plant as described herein, e.g. in a winter oilseed rape or a winter wheat plant, provides a means to overcome the most significant bottleneck in accelerating genetic gain in such crops.

Furthermore, the results yielded by the present methods provide researchers and breeders with strategies to accelerate crop cycle time and genetic gain; and provide operational flexibility and scalability that traditionally were not possible. As such, the methods described herein may further reduce generation time, which enables rapid advancement of improved genotypes to meet specific agronomic goals. Regarding WOSR, for example, decreasing crop cycle time can accelerate development of WOSR pedigrees having an improved oil profile, increased disease resistant, higher yield, and/or shatter-proof pods. Similarly, in wheat, for example, decreasing crop cycle time can accelerate the development of wheat pedigrees having increased yield and improved climate resilience.

Plants useful in accordance with the present invention may include, but are not limited to, wheat, barley, rye, oat, oilseed rape, bok choy, cabbage, cauliflower, collards, broccoli, brussels sprouts, kale, kohlrabi, rutabagas, turnips, sugar beet, corn, soybean, and cotton. In specific embodiments, the plant may be a winter oilseed rape plant or a winter wheat plant.

The methods described herein alter normal plant phenology and decouple competency of flowering from exposure to vernalization conditions by using far-red light signals to induce a flowering response in a temperature agnostic manner. The strength and speed of the flowering response can be further modulated by varying additional parameters as described herein. For example, utilizing the described methods, in conjunction with temperature, daylength, light intensity, FR duration, and R:FR ratio, may result in at least 1.5, 1.6, 1.7, 1.8, 1.9, 2, 3, 4, or more growth cycles per year across diverse germplasms.

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology, plant development, and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, the term “photoperiod” means the duration of light exposure in a 24-hour period.

As used herein, the term “winter annual” or “winter annuals” refers to plants that germinate in autumn or winter, live through the winter, and then bloom in winter or spring. Winter annuals are known to require vernalization in order to flower. For example, winter oilseed rape (WOSR) is a winter annual planted in the fall that requires vernalization during winter to flower during the following spring. Spring oilseed rape, also known as Canola, is an example of a Brassica napus plant that does not require vernalization to flower.

As used herein, the term “far-red light” (FR) describes light having a wavelength between 700 nm to 750 nm. For example, light at a wavelength of 730 nm.

The transition to flowering depends on the activity of apical meristems. As used herein, “flowering response” can refer to a plant's meristem gaining competency to flower. The completion of a flowering response is reflected by flower bud formation leading to flower development.

As used herein, a “flowering time” can refer to the occurrence of flowering formation, e.g. when floral buds are visible from the top view (bud visible). Floral bud visibility indicates commitment and/or completion of flowering response and floral development with a first open flower occurring about 7 to 10 days after bud become visible, as in the case of WOSR.

As understood in the art, “seed maturation” is a key period in a plant's lifecycle. Events occurring during maturation include storage reserve deposition, desiccation, dormancy induction, seed coat formation, and protective compound synthesis.

As used herein, “synergistic” or “synergistically” refers to a combination of two growth conditions, e.g. temperature and far-red light treatment, as described herein that more effectively accelerates flowering than the respective growth conditions alone.

As used herein, typical, or regular “vernalization” refers to incubating plants at a temperature from about 4 to about 6° C. for about 8 weeks under varied photoperiod. In contrast, “unvernalized” refers to plants that have not been exposed to such prolonged cold temperature treatment, e.g. plants grown at temperatures higher than 10° C.

As used herein, “vegetative propagation” refers to any form of asexual reproduction occurring in plants in which a new plant grows from a fragment of the parent plant. Non-limiting examples of vegetative propagation methods include tissue culture and division.

As used herein, the term “isogenic” means genetically uniform, whereas non-isogenic means genetically distinct.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

### Example 1. Micrografting and Macrografting of Winter Oilseed Rape

WOSR (Brassica napus) is an allotetraploid developed from hybridization of Brassica rapa and Brassica oleracea. The WOSR genome is highly duplicated with 9 copies of annotated FLC, with at least 5 having the ability to delay flowering in Arabidopsis (See Tadege, M. et al. Plant J 28, 545-553 (2001)). The multiple FLC paralogs suggests that the FLC-mediated flowering suppression is more tightly regulated in WOSR, making significantly accelerating flowering time more challenging than in small genome plant species. In this example, a macrografting technique was used to determine whether FLC repression by vernalization is absolutely required for WOSR to gain competency to flower.

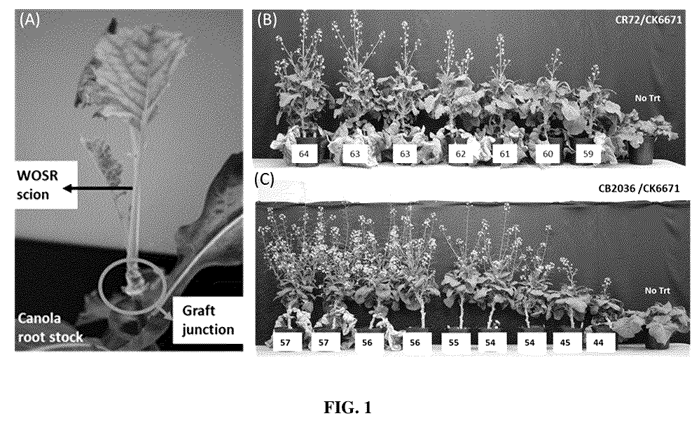

In particular, unvernalized 3-week-old WOSR scions were grafted onto Canola root stock (spring variety of OSR, no cold exposure required for flowering). Surprisingly, these grafting experiments demonstrated that WOSR can flower without vernalization, indicating mobile flowering signals from the root stock are sufficient to promote flowering in unvernalized WOSR scions (FIG. 1).

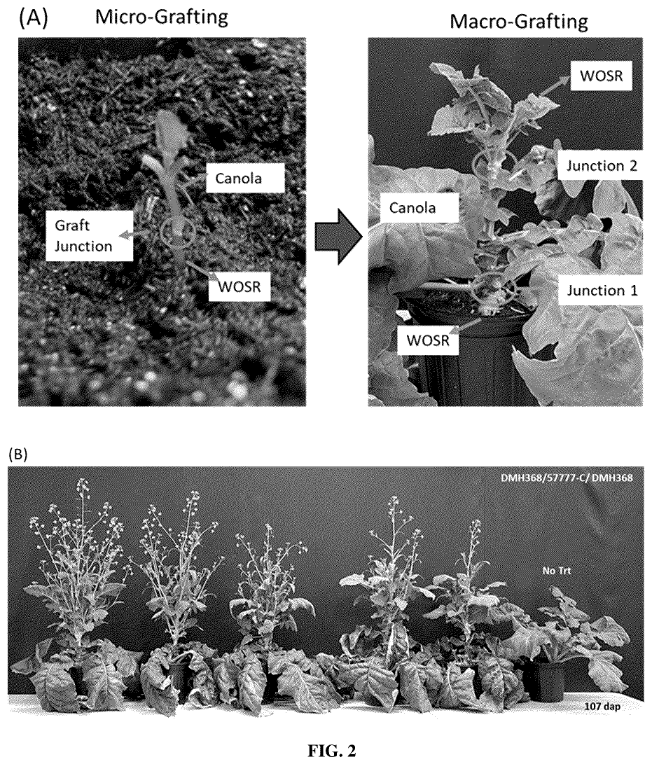

To further validate these results and to investigate whether unvernalized WOSR root might convey any negative signal to prevent WOSR scion from flowering, a double grafting experiment was carried out. In particular, a first graft (i.e. a micrograft) where the WOSR root is joined with canola shoot at the hypocotyl was completed a few days after germination. After the micrografted plant reached the bolting stage, a second graft (i.e. a macrograft) was completed where a 3-week-old unvernalized WOSR scion shoot was grafted onto the bolting stem from the micrografted plant (FIG. 2). Consistent with the initial results, WOSR scions were able to proceed to flower in the double grafted plants, indicating flowering signal from a canola rosette was enough to drive flowering; and signals, if any, from the unvernalized WOSR root can be bypassed. This example demonstrates that it is possible to induce WOSR flowering without the need of prolonged cold treatment and accelerate WOSR flowering time in a germplasm-independent fashion.

### Example 2. FR Accelerates Winter and Spring OSR Flowering Under Standard Growth Temperature

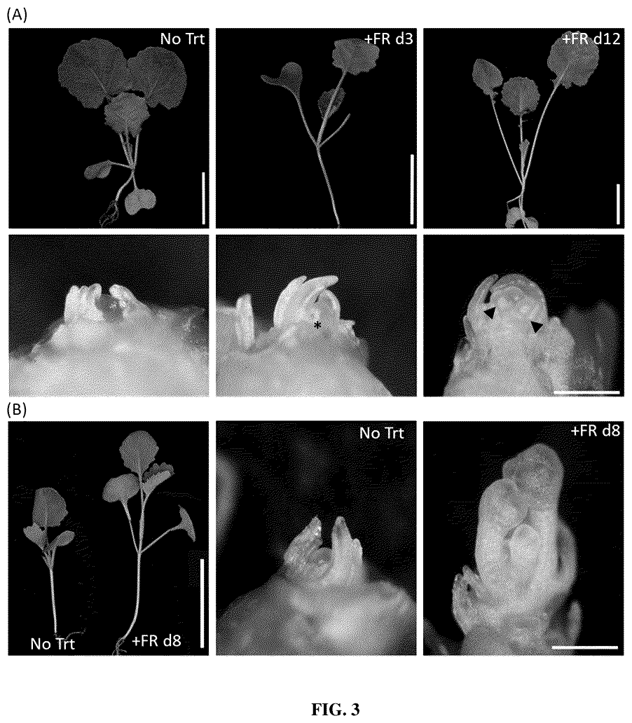

To determine whether far-red light (FR) treatment can accelerate oilseed rape flowering under standard growth conditions, FR treatment was started on day three after planting for both winter and spring oilseed rape plants. Plants were treated under FR LED with a peak wavelength around 730 nm in addition to broad spectrum supplemental lights. Both the FR and the broad spectrum supplemental lights were set to 22 hr photoperiod in this example. The resulting R:FR ratio was between 0.1 to 0.4. Images of meristems were obtained. Enlarged meristem indicative of reproductive transition after 3 days under FR was observed for WOSR pedigree MSP12 (FIG. 3A; +FR d3, asterisk). Floral primordia were also initiated after 12 days under FR (FIG. 3A; +FR d12, arrowheads). 15-day-old no treatment (No Trt) plants of the same age as +FR d12 were also imaged as a comparison. Flowers of spring oilseed rape (a.k.a. canola) are fully differentiated after 8 days under FR (FIG. 3B; +FR d8). 11-day-old no treatment (No Trt) plants of the same age were also imaged as a comparison. In particular, FIG. 3A demonstrates that reproductive meristem tissue is produced in WOSR following FR treatment; and flower development is accelerated in both winter and spring oilseed rape plants following FR treatment.

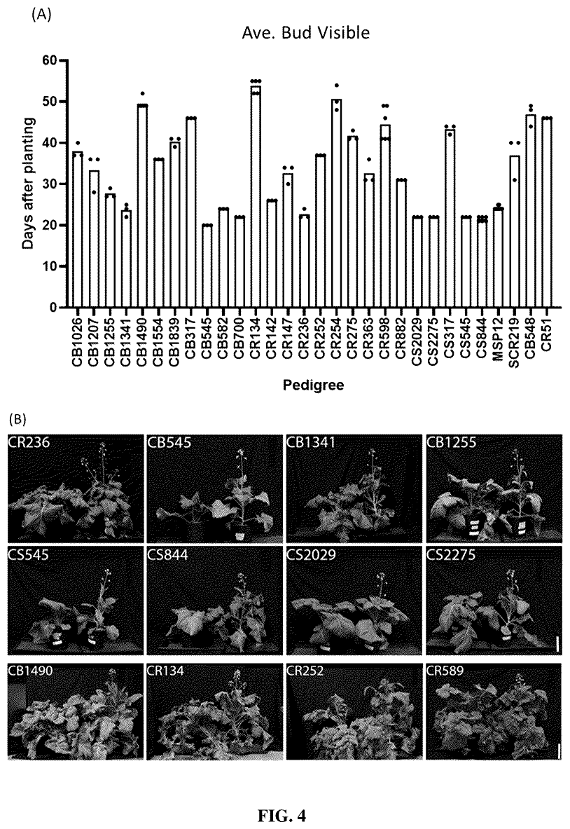

Similar experiments were carried out using 30 individual WOSR pedigrees, representing diverse germplasm pools. The results from these experiments were consistent with those described above. See FIG. 4. That is, treatment of juvenile plants with FR light can induce flowering without the need for prolonged low temperature treatment (vernalization treatment) in a germplasm-independent fashion.

### Example 3. FR Activates FT Expression Without Reduction of FLC Expression or Epigenetic Silencing of FLC Loci

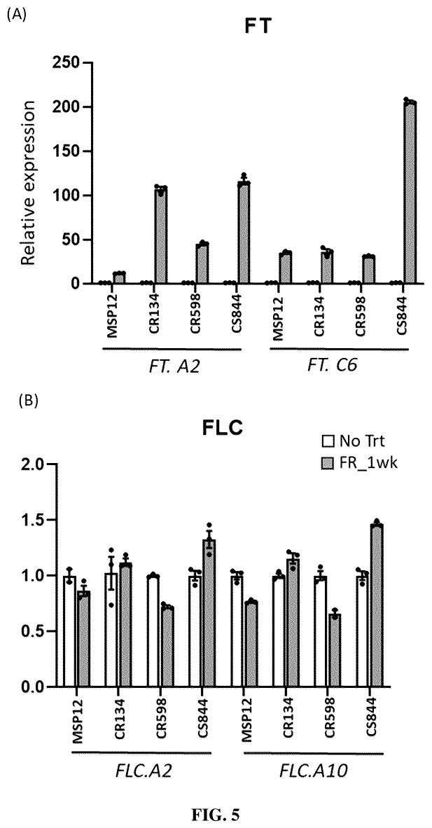

Quantitative real-time PCR was used to analyze expression of two FT or FLC paralogs in four representative WOSR pedigrees. In particular, cotyledons from four representative WOSR pedigrees were harvested after 1 week with or without FR treatment. This expression analysis demonstrated that flowering promoter FT can be dramatically upregulated in response to FR treatment without any significant reduction of FLC across diverse WOSR pedigrees. See FIG. 5, which shows that flowering activators FT. A2 and FT. C6 are significantly upregulated in all WOSR pedigrees (and more than 100-fold in select WOSR pedigrees). Conversely, expression levels of flowering inhibitors FLC.A2 and FLC.A10 show no significant change based on FR treatment.

Histone modification H3K27me3 at the FLC loci is believed to be required to induce flowering in vernalization dependent plants. That is, prolonged cold exposure induces stable epigenetic silencing of FLC via histone modification H3K27me3; and this silencing is required for flowering. See, e.g. Baulcombe DC, et al. Cold Spring Harb Perspect Biol. 2014 Sep. 2;6(9):a019471 (2014). However, the results described herein demonstrate that FR-treated plants produced floral buds about 20-45 days after planting; and FLC transcription reduction and epigenetic silencing is not required for WOSR to flower following FR treatment.

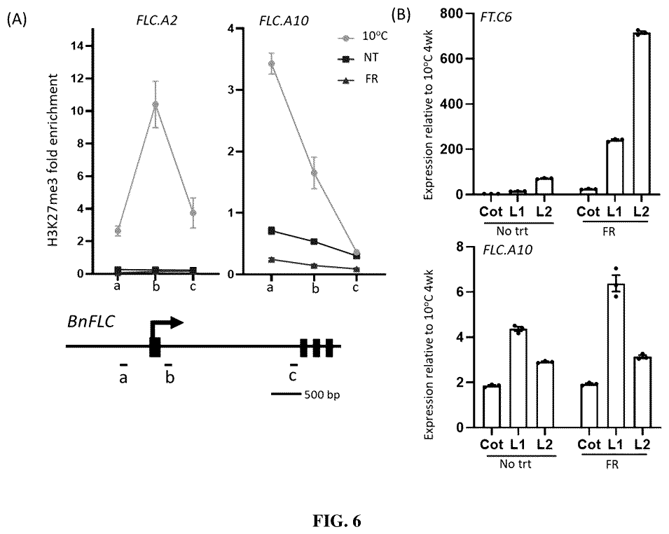

In particular, chromatin immunoprecipitation followed by quantitative PCR of H3K27me3 levels at two representative WOSR FLC paralogs from CR134 plants treated with 10° C. for 4 weeks (10° C.), no treatment (No Trt), and FR-treated (FR) for 14 days starting on day 3 after planting (wherein the shoot apical meristem has undergone floral transition) were analyzed (FIG. 6). Contrary to current paradigm, these experiments demonstrated that epigenetic silencing at WOSR FLC loci is not required for WOSR to flower with FR. That is, epigenetic silencing at representative WOSR FLC loci remains low in plants treated with FR as described herein, similar to No Trt plants.

### Example 4. Low Temperature Incubation and FR Treatment Create Synergistic Effects Toward Accelerating Flowering

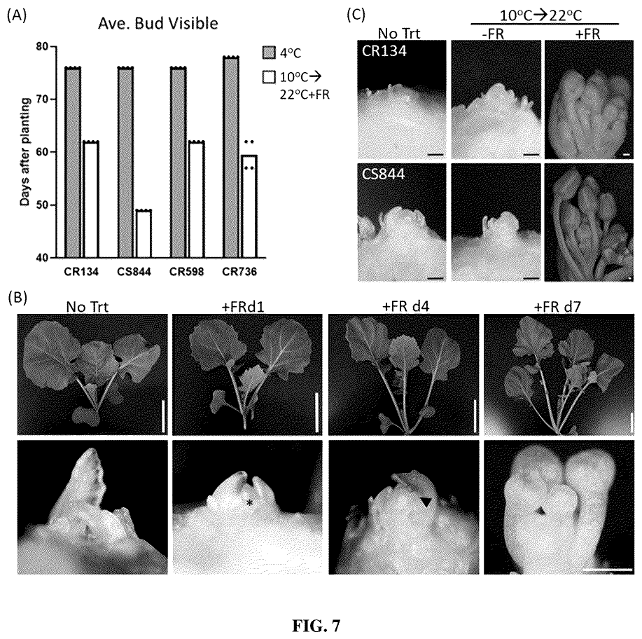

The effect of FR application during low temperature treatment was evaluated. It was found that a simultaneous reduction of FLC by low temperature along with upregulation of FT by FR creates a synergistic effect toward flowering time acceleration. In particular, 2-week-old plants were incubated at low temperature for 4 weeks followed by treatment with FR after returning to warm temperature (10° C. →22° C.+FR) and control plants were incubated at 4° C. (conventional vernalization protocol). FIG. 7 shows the average bud visible time of 4 representative pedigrees was 7-9 weeks compared to 11 weeks using conventional vernalization protocol (4° C.). These results demonstrate that adding FR after vernalization accelerates flowering compared to conventional vernalization protocols (FIG. 7A and B). Moreover, adding FR (+FR) after low temperature treatment shows synergistic effects in flowering acceleration (FIG. 7C).

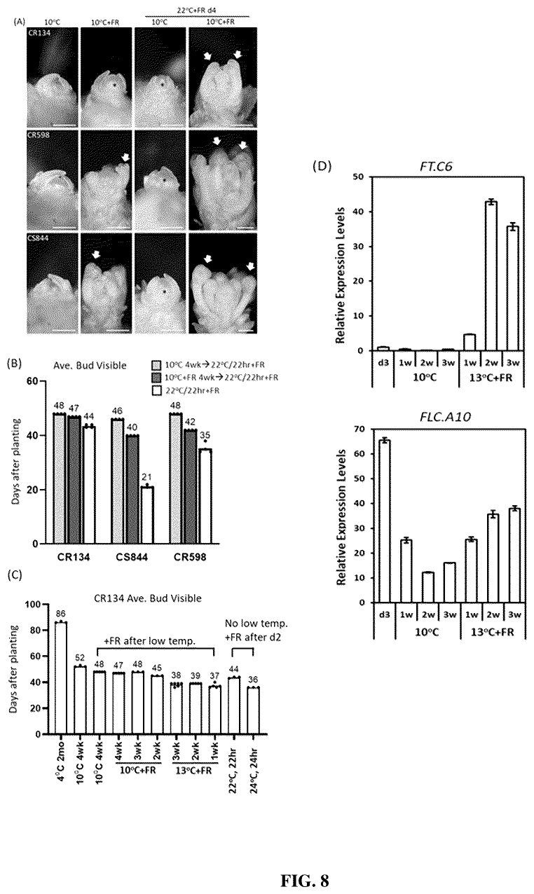

FR treatment across various temperatures was further evaluated. The reproductive transition or flower formation of three representative pedigrees of WOSR (CR134, CR598 and CS844) was observed under various conditions, including 10° C. without FR and 10° C. with FR. Reproductive transition or flower formation of all three pedigrees is visible after 4 weeks under 10° C.+FR (FIG. 8A; asterisk and arrows). These results demonstrate that adding FR during 10° C. accelerates flowering in WOSR. Furthermore, adding FR after returning plants to 22° C. leads to reproductive transition four days after returning to 22° C. even in plants incubated at 10° C. without FR (FIG. 8A; asterisks). On the same day, more advanced flower development seen in plants treated with FR during the 10° C. incubation (arrows). As shown in FIG. 8B-C, supplementing FR during low temperature treatment further reduces bud visible time; and continuous FR treatment with background lighting for 24 hr daily shows the most significant reduction in bud visible time. Incredibly, these results further demonstrated that the average bud visible time of WOSR can be drastically reduced by adding FR two days after planting without low temperature treatment (FIG. 8C). The FR treatment methods described herein can accelerate flowering in a temperature-agnostic fashion. The FR treatment protocols described herein are therefore applicable across a wide range of growth temperatures, providing operational flexibility in WOSR breeding programs.

### Example 5. FR Treatment Coupled With Increasing Photoperiod

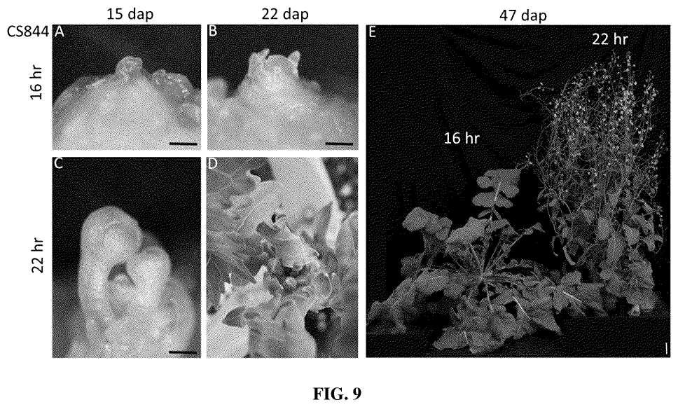

The effect of FR application across varying photoperiods was also evaluated. Specifically, WOSR pedigrees were grown under 16 hr photoperiod and 22 hr photoperiod, respectively. Under 16 hr photoperiod the representative WOSR pedigree CS844 produced floral buds between 55 and 60 days after planting (dap). In stark contrast, CS844 produced floral buds between 22-21 dap under 22 hr photoperiod (FIG. 9). As such, FR treatment coupled with >16 hr photoperiod can further accelerate flowering.

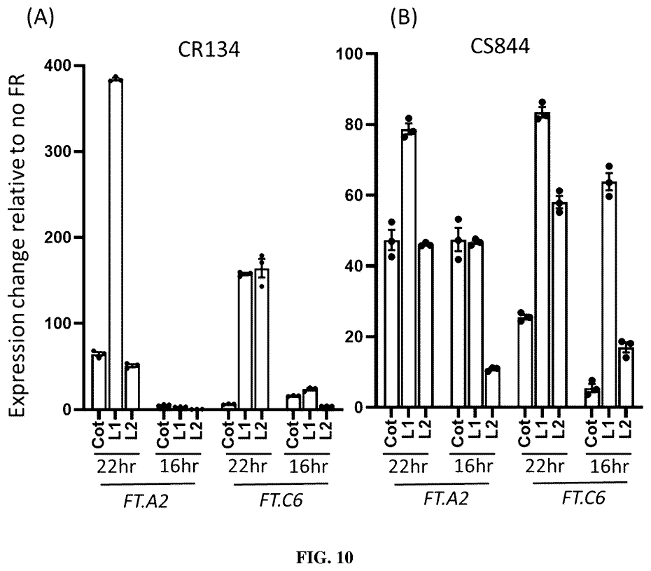

Quantitative real-time PCR analysis further demonstrated that expression of FT paralogs A2 and C6 from WOSR are significantly upregulated when FR treatment is applied under >16 hr photoperiod (FIG. 10).

### Example 6. Vegetatively Propagated Plants Derived From FR-Treated WOSR Continue to Flower

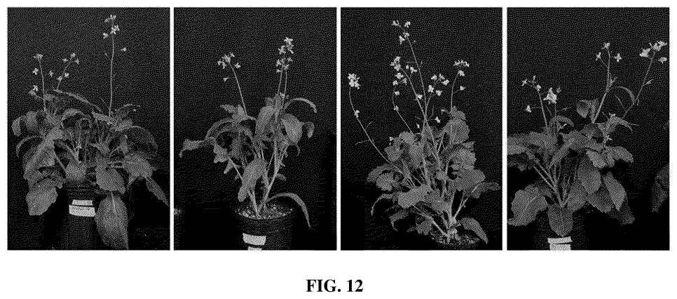

Flowering of vegetatively propagated plantlets, derived from FR-treated WOSR plants that have not been exposed to vernalization conditions, is demonstrated in the following example. In particular, cuttings from FR-treated WOSR inflorescences were treated with rooting hormones at the cut site then inserted into peat moss-based plugs until roots were initiated. The resulting plantlets were transplanted into nursery pots with peat moss-based soilless medium until flowering. Vegetative propagation from FR-treated WOSR continued to flower without reverting back to vegetative development, consistent with the finding that FLC reactivation occurs during early embryogenesis (Choi, J. et al. Plant J 57, 918-931 (2009)). Flowering vegetatively propagated plants from four representative pedigrees are shown in FIG. 12 (representative images were taken 3 to 4 weeks after newly formed floral buds are visible from the top). As such, the methods using FR described herein not only allow for inducing a flowering response in a temperature-agnostic fashion, but these advantages are also maintained in vegetatively propagated plantlets derived therefrom.

### Example 7. FR Supplementation in Winter Wheat Accelerates Winter Wheat Cycle Time

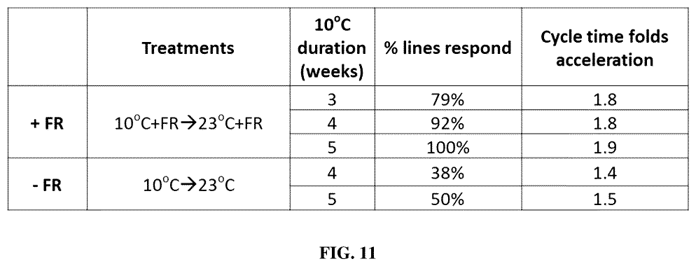

The methods described herein can also be applied to other winter annuals, including winter wheat. Winter wheat lines were treated with different durations of 10° C. with or without FR supplement in addition to broad spectrum supplemental lights then moved to 23° C. standard growth conditions with or without FR treatment. A 22 hr photoperiod was used for all conditions. In total 32 diverse elite winter wheat lines were represented in these experiments. Application of FR treatment to diverse elite winter wheat lines resulted in an almost two-fold acceleration in winter wheat cycle time (cycle time is calculated based on seed-to-seed time compared to conventional methods). Moreover, when winter wheat lines were treated with FR at 10° C. for 5 weeks, 100% of the elite lines flowered (FIG. 11).

### Example 8. Effect of FR Light Treatment Initiation at Varying Plant Age

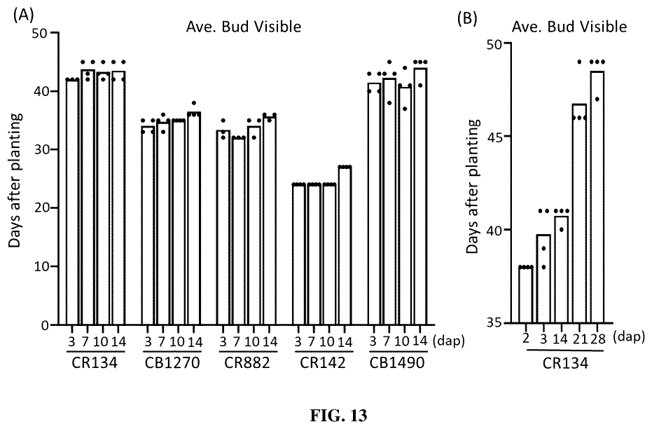

To determine whether far-red light (FR) treatment can accelerate or bypass vernalization requirement of winter oilseed rape flowering when initiated at different ages of seedlings, FR treatment was initiated on plants from 2 days after planting to 28 days after planting. Plants were treated under FR LED with a peak wavelength around 730 nm in addition to broad spectrum white light LED supplemental lights. Average floral bud visible time was evaluated. In FIG. 13, Panel A. Seedlings were germinated at 22° C., 22 hr photoperiod under broad spectrum LED set to 300 μmol m⁻² s⁻¹. Seedlings at different ages (days after planting, dap) were then supplemented with FR light set to 22 hr photoperiod until floral buds were visible from the top. All WOSR plants produced visible buds and flowers without vernalization. Observation was repeated with recalcitrant germplasm (CR134) in FIG. 13 (Panel B), where Seedlings were germinated at constant 24° C. with constant light (24 hr) set to same intensity as in Panel A. Seedlings at different ages (days after planting, dap) were then supplemented with FR light set at 24 hr photoperiod, same as broad spectrum light, until floral buds were visible from the top. These results demonstrate that FR treatment can enable WOSR to bypass vernalization requirements and accelerate flowering when treated as late as 28 days after planting.

### Example 9. Effect of R/FR Ratio on FR Driven Flowering Acceleration of WOSR

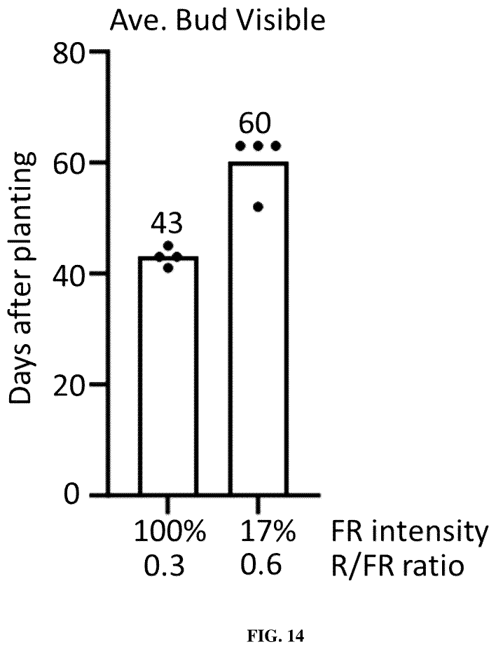

To understand light quality effect on FR treatment-driven WOSR flowering acceleration, we tested different R/FR ratio's impact on WOSR vernalization-free flowering. To achieve different R/FR ratio, FR intensity was adjusted to 17% of standard treatment condition (100% FR), while red light intensity from background light remained constant. With 17% of FR intensity, we achieved R/FR ratio of 0.6, whereas the control (100% FR intensity) R/FR ratio is 0.3 in this experiment. These results demonstrate that increasing R/FR ratio from 0.3 to 0.6 significantly delayed floral bud visible time by 17 days in the absence of vernalization, as shown in FIG. 14.

### Example 10. FR Treatment Effect is Photoperiod-Dependent and FR Treatment Does not Need to be Continuous

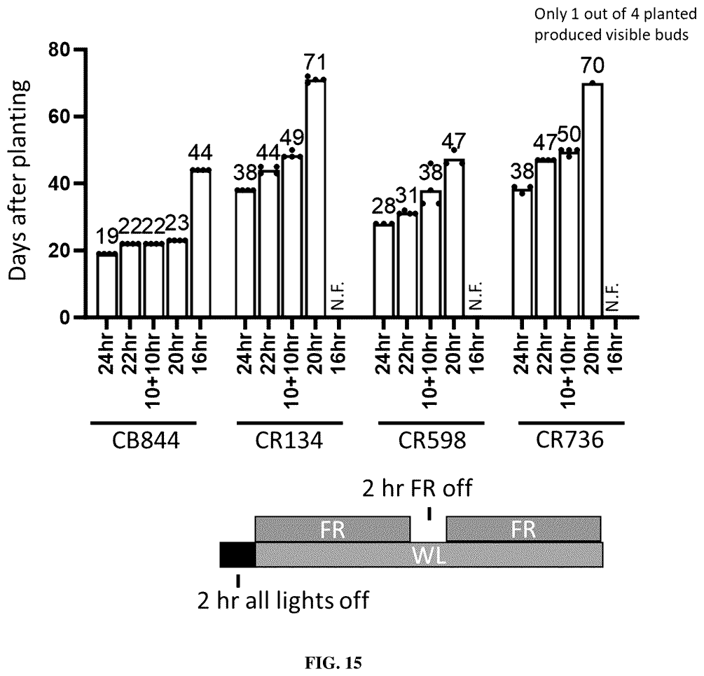

To understand the role of photoperiod in FR-mediated vernalization-free flowering acceleration, representative pedigrees were tested under different photoperiods as shown in FIG. 15. Across all four pedigrees, plants flowered sooner with longer photoperiod. Some pedigrees are more sensitive to photoperiod than others. For example, in CR736 and CR134, flower acceleration was attenuated when grown in 20 hour photoperiod and no flowering was observed under 16 hour photoperiod till experiment termination at >70 days after planting. For all treatments, except treatment 10+10, WL (white light LED and FR are synchronized, meaning same duration of light illumination for WL and FR). To test whether FR treatment must be continuous, 10+10 treatment was included (diagramed in FIG. 15, Panel B), where the background white light LED was set to 22 hour photoperiod and FR supplemental light was set as 10 hour+10 hour with 2 hours break in the middle. As shown in FIG. 15 (Panel A), FR light treatment does not have to be continuous to be effective in driving vernalization-free flowering acceleration. Shorter photoperiod of FR illumination (10 hrs), can still enable vernalization-free flowering acceleration. However, in comparing 22 hr (22 hr WL+22 hr FR) and 10+10 treatment, it indicated that shorter duration of FR treatment can slow down flower acceleration when supplemental WL photoperiod remained the same.

### Example 11. FR Treatment Effect on Accelerating Wheat Flowering

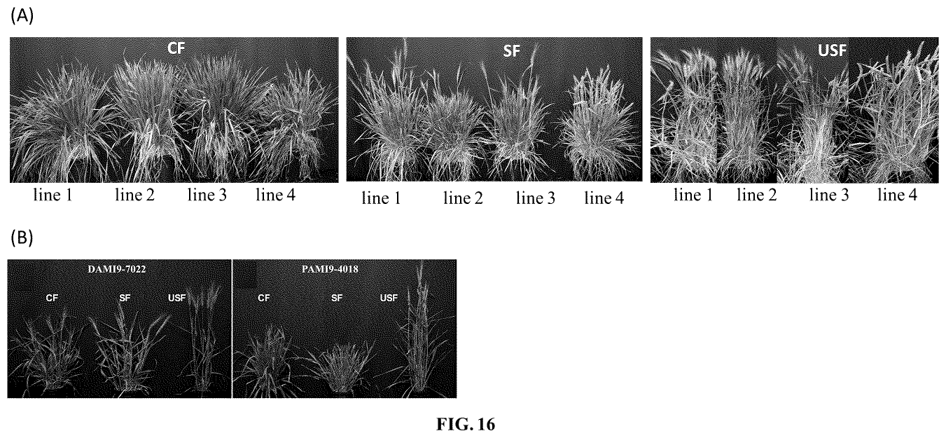

The methods described herein can also enable vernalization-free flowering in winter wheat. Representative winter wheat lines that represent diverse germplasm pools were tested. Under control conditions, no sign of flowering was observed from the tested winter wheat lines. Under long day conditions (22 hour day/night photoperiod), when supplemented with Far red light, all four tested lines have synchronized flowering. In contrast, under long day conditions alone, only line 4 has synchronized flowering. The other 3 lines had non-synchronized and sparsely flowering (FIG. 16; Panel A). Similar treatment was also performed on spring wheat lines, it was observed that under long day plus FR conditions, spring wheat flowering and maturation can be further accelerated (FIG. 16; Panel B).

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations can be applied to the materials and methods described herein without departing from the concept, spirit, and scope of the invention. All such similar substitutes and modifications apparent to those skilled in the art are deemed to be within the spirit, scope and concept of the invention as defined by the appended claims.

1. A method for decreasing flowering time in a plant, the method comprising:
(a) obtaining a juvenile plant; and
(b) treating the juvenile plant with far-red light to induce a flowering response in said plant.

2. The method of claim 1, wherein said treating:
is carried out when the plant is from about 1 day to about 7 weeks from germination;
is carried out at a temperature of from about 4° C. to about 30° C.;
is carried out for about 1 to about 7 weeks;
is carried out at a temperature of about 10° C. for about 1 to about 5 weeks;
is carried out in the presence of visible light;
comprises growing the plant under a photoperiod from 16 hr to 24 hr;
is carried out at a red light to far-red light ratio between 0.01 to 0.8; or
comprises applying far-red light to the plant for from about 1 hr to about 24 hr per day prior to flowering.

3. (canceled)

4. The method of claim 2, wherein said treating is carried out at a temperature:
from about 6° C. to about 15° C.; or
from about 15° C. to about 30° C.

5-8. (canceled)

9. The method of claim 1, wherein:
the method further comprises growing the plant under far-red light;
the far-red light comprises an intensity of about 50 μmoles/m·s to about 800 μmoles/m·s;
the far-red light is applied continuously;
the far-red light is applied non-continuously;
the plant is treated with far-red light for about 7 to about 60 days prior to flowering;
said plant is a vernalization dependent plant;
said plant is a vernalization independent plant;
the flowering response is induced without vernalization;
the flowering response is initiated in less than about 45 days from germination;
said plant is a plant selected from the group consisting of wheat, barley, rye, oat, oilseed rape, bok choy, cabbage, cauliflower, collards, broccoli, brussels sprouts, kale, kohlrabi, rutabagas, turnips, sugar beet, corn, soybean, and cotton;
said plant is a winter oilseed rape plant;
said plant is a winter wheat plant;
the method further comprises allowing the plant to develop at least a first flower; or
the method further comprises harvesting a seed from the plant.

10. The method of claim 9, wherein growing:
the plant under far-red light accelerates plant development as compared to a control plant lacking said treatment;
the plant under far-red light accelerates seed maturation as compared to a control plant lacking said treatment;
is carried out at a temperature of from about 10° C. to about 34° C.; or
the plant under far-red light is carried out at a temperature of about 10° C. to about 25° C.

11-14. (canceled)

15. The method of claim 2, wherein the photoperiod is at least 20 hr.

16-25. (canceled)

26. The method of claim 9, wherein the flowering response is initiated in less than about 7 days from germination.

27-30. (canceled)

31. The method of claim 9, the method further comprising crossing the plant with itself or a second non-isogenic plant.

32. (canceled)

33. A method of bypassing vernalization dependent flowering in a plant, the method comprising:
(a) obtaining a juvenile plant; and
(b) treating the juvenile plant with far-red light to induce a flowering response in said plant;
wherein said plant is a vernalization dependent plant and wherein said treating is carried out without vernalization prior to said flowering response.

34. The method of claim 33, wherein:
the method further comprises vegetatively propagating said plant;
the flowering response results in at least a first floral bud forming less than about 60 days after germination of said plant;
the method further comprises harvesting a seed resulting from said flowering response; or
the method further comprises crossing the plant with itself or a second plant to produce a progeny plant.

35-37. (canceled)

38. The method of claim 34, further comprising harvesting a seed resulting from said crossing.

39. A method of accelerating crop cycle time in a plant, the method comprising:
(a) obtaining a juvenile plant;
(b) treating the juvenile plant with far-red light to induce a flowering response in said plant; and
(c) allowing the plant to develop at least a first floral bud;
wherein the first floral bud is visible in less than about 60 days from germination.

40. The method of claim 39, wherein:
the method further comprises growing the plant under far-red light, wherein growing the plant under far-red light accelerates plant development;
the method further comprises harvesting a seed from the plant; or
said plant is a vernalization dependent plant.

41. The method of claim 40, wherein the method comprises growing the plant under far-red light following development of at least the first floral bud, wherein growing the plant under far-red light accelerates seed maturation.

42. (canceled)

43. (canceled)

44. A method for decreasing seed to seed cycle time in a plant, the method comprising:
(a) obtaining a juvenile plant;
(b) exposing the juvenile plant to a temperature from about 4° C. to about 15° C. to induce a flowering response in said plant; and
(c) growing the plant at a temperature from about 16° C. to about 34° C.

45. The method of claim 44, wherein:
step (b) further comprises treating the plant with far-red light;
step (c) further comprises treating the plant with far-red light; or
steps (b) and (c) further comprise treating the plant with far-red light.

46. The method of claim 45, wherein treating the plant with far-red light accelerates plant development or seed maturation as compared to a control plant lacking said treatment.

47. The method of claim 44, wherein:
exposing the plant to a temperature from about 4° C. to about 15° C. is carried out for about 1 to about 7 weeks;
the plant is exposed to a temperature from about 10° C. to about 15° C. for about 4 weeks; or
growing the plant is carried out at a temperature of about 22°.

48. (canceled)

49. (canceled)
